(19) United States
(12) Patent Application Publication
(10) Pub. No.: US 2025/0090008 A1
(43) Pub. Date: Mar. 20, 2025

(54) CAPSULE ENDOSCOPE AND CONTROL METHOD THEREFOR

(71) Applicants: Ankon Medical Technologies(Shanghai) Co., LTD, Shanghai (CN); ANX IP HOLDING PTE. LTD., SINGAPORE (SG)

(72) Inventor: Xiaodong DUAN, Plano, TX (US)

(73) Assignees: Ankon Medical Technologies(Shanghai) Co., LTD, Shanghai (CN); ANX IP HOLDING PTE. LTD., SINGAPORE (SG)

(21) Appl. No.: 18/725,809

(22) PCT Filed: Dec. 29, 2022
(86) PCT No.: PCT/CN2022/143591

§ 371 Date: Jul. 1, 2024

(30) Foreign Application Priority Data
Dec. 31, 2021 (CN) 202111673349.8

(51) Int. Cl. A61B 1/04; A61B 1/00; A61B 1/12

(52) U.S. Cl. CPC A61B 1/041; A61B 1/00064; A61B 1/128

(57) ABSTRACT

The present invention provides a capsule endoscope and a control method therefor. The capsule endoscope comprises a temperature sensing element, a heat-generating component, and a circuit processing component, where the temperature sensing element is used for measuring the temperature of the heat-generating component inside the capsule endoscope, the temperature signals measured by the temperature sensing element are transmitted to the circuit processing component, which controls the heat-generating component of the capsule endoscope to switch between a normal working mode and a temperature control mode based on the measured temperature signals. According to the capsule endoscope and the control method therefor of the present invention, local overheating of the capsule endoscope during high-power-consumption operation can be effectively avoided.

### FIELD OF INVENTION

The present invention relates to a miniature medical robot, and more particularly to a capsule endoscope and a control method therefor.

### BACKGROUND

Capsule endoscope, depending on its high reliability and safety, has become an effective device for the diagnosis of gastrointestinal diseases and has obtained high recognition in international medical device field. A capsule endoscope includes a CMOS (Complementary Metal Oxide Semiconductor) image sensor, an optical system, a battery, a transmission circuit and an antenna. Images of the gastrointestinal tract of a human subject are formed on the surface of the CMOS image sensor through the optical system. The CMOS image sensor converts the optical signals into electrical signals, which are then modulated and amplified by the transmission circuit and transmitted through the antenna. These signals are received by an external receiving device and subsequently displayed on a display device. Based on the displayed images, a physician can make a diagnosis of gastrointestinal diseases for the subject in a state of painless and non-invasive gastrointestinal peristalsis.

During the examination process with the capsule endoscope, there is an increasing demand for functions such as high-frequency photography, video recording, and high-precision positioning. However, functions such as high-frequency photography, video recording, and high-precision positioning not only consume a significant amount of power, but also generate considerable heat in the corresponding electronic components. If the accumulated heat is not managed properly, it can lead to performance degradation or even damage to the corresponding electronic components.

Therefore, there is a need for a capsule endoscope and its control method that can prevent localized overheating.

### SUMMARY OF THE INVENTION

In order to solve the problems in the prior art, the present invention provides the following solution:

A capsule endoscope, comprising a temperature sensing element, a heat-generating component, and a circuit processing component, wherein the temperature sensing element is used for measuring the temperature of the heat-generating component inside the capsule endoscope, the temperature signals measured by the temperature sensing element are transmitted to the circuit processing component, which controls the heat-generating component of the capsule endoscope to switch between a normal working mode and a temperature control mode based on the measured temperature signals.

Preferably, the heat-generating component includes a plurality of components, and the temperature sensing element respectively measures the temperatures of the heat-generating components in real-time.

Preferably, the heat-generating component includes an image acquisition component and/or a circuit processing component.

Preferably, the temperature sensing element measures the temperatures of both the image acquisition component and the circuit processing component in real-time.

According to the technical solution of the present invention, when the capsule is located in a stomach and the temperature of the heat-generating component is above a first threshold, the heat-generating component enters a first temperature control mode. When the temperature of the heat-generating component is equal to or below a second threshold, the heat-generating component is switched to the normal working mode.

The first threshold temperature is below the tolerance limit temperature of the heat-generating component, to prevent overheating of the heat-generating component during high-frequency photographing, video recording, and high-precision positioning and other operations, which could cause damage.

Preferably, the first threshold may be 60° C.-70° C. Further preferably, the first threshold may be 60° C.-65° C.

The first temperature control mode is preferably a power consumption reduction mode. Preferably, in the first temperature control mode, the power consumption of the heat-generating component is reduced to 6%-50% of the normal working mode. Further preferably, in the first temperature control mode, the power consumption of the heat-generating component is reduced to 20%-40% of the normal working mode.

When the heat-generating component is the image acquisition component, the power consumption reduction method for the image acquisition component may be lowering the frame rate or switching from video mode to photo mode, etc. Preferably, the image acquisition component reduces power consumption by lowering the frame rate.

When the heat-generating component is the circuit processing component, the power consumption reduction method for the circuit processing component may be reducing position detection accuracy, lowering transmission power, or not performing computations on the storage. Preferably, the circuit processing component reduces power consumption by increasing the time interval between position detections or not performing computations on the storage. Optionally, the power consumption of the image acquisition component and the circuit processing component may be reduced by lowering the photographing rate.

In the present invention, the second threshold temperature is lower than the first threshold temperature. Preferably, the second threshold is 9° C.-15° C. lower than the first threshold. Further preferably, the second threshold is 10° C.-12° C. lower than the first threshold. The temperature values set by the first threshold and the second threshold can prevent local overheating, and avoid frequent switching of the working mode, thus ensuring stable and reliable operation of the capsule endoscope.

According to the technical solution of the present invention, if the temperature of the heat-generating component remains above the second threshold after continuously operating in the first temperature control mode for a first specific time, the heat-generating component enters the third temperature control mode. When the temperature of the heat-generating component is equal to or below the second threshold, the heat-generating component is switched to the normal working mode.

The first specific time is 30 s-60 s. Preferably, the first specific time is 60 s.

In the present invention, the third temperature control mode may be either a power consumption reduction mode or an off mode.

Optionally, the third temperature control mode is a power consumption reduction mode, where the power consumption of the heat-generating component is reduced to 1%-3% of the normal working mode.

Optionally, the third temperature control mode is an off mode.

When the third temperature control mode is an off mode, while any heat-generating component enters the off mode, the capsule endoscope enters a sleep state.

Preferably, after entering the sleep state, the capsule endoscope is cooled in a cooling medium. The capsule endoscope is fully immersed in the cooling medium.

Preferably, before entering the sleep state, the working position and orientation information of the capsule endoscope are recorded, and then the capsule endoscope enters the sleep state.

Optionally, the cooling medium may be gastric juice. Optionally, if the capsule endoscope is not fully immersed in the gastric juice, it can be controlled to fully immerse in the gastric juice via an external magnetic control device.

Optionally, after entering the sleep state, when the temperatures of all heat-generating components are equal to or below the second threshold, the heat-generating components are switched to the normal working mode, and the capsule endoscope enters the normal working state.

Preferably, the capsule endoscope returns to the original working state and continues working based on the working position and orientation information recorded before entering the sleep state.

Preferably, if the temperature of any heat-generating component remains above the second threshold after continuously operating in the first temperature control mode for the first specific time, the working position and orientation information of the capsule endoscope are recorded, and then the capsule endoscope enters the sleep state. The capsule endoscope in the sleep state is fully immersed in the gastric juice for cooling or controlled to fully immerse in the gastric juice for cooling via an external magnetic control device. When the temperatures of all heat-generating components are equal to or below the second threshold, the capsule endoscope is activated, switching the heat-generating components to the normal working mode. The capsule endoscope returns to the original working state and continues working based on the working position and orientation information recorded before entering the sleep state.

According to the technical solution of the present invention, when the capsule is located in the intestinal tract and the temperature of the heat-generating component is above the first threshold, the heat-generating component enters a second temperature control mode. When the temperature of the heat-generating component is equal to or below the second threshold, the heat-generating component is switched to the normal working mode.

When the capsule is located in the intestinal tract, the frame rate, position detection accuracy, etc., set up for the capsule in the normal working mode are lower than those in the stomach examination. Preferably, when the capsule is located in the intestinal tract, the second temperature control mode is preferably a power consumption reduction mode. Preferably, in the second temperature control mode, the power consumption of the heat-generating component is reduced to 50%-80% of the normal working mode.

If the temperature of the heat-generating component remains above the second threshold after continuously operating in the second temperature control mode for a second specific time, the heat-generating component enters a fourth temperature control mode. When the temperature of the heat-generating component is equal to or below the second threshold, the heat-generating component is switched to the normal working mode.

The second specific time is 1 min-10 min. Preferably, the second specific time is 3 min-7 min.

In the present invention, the fourth temperature control mode is preferably a power consumption reduction mode. Preferably, in the fourth temperature control mode, the power consumption of the heat-generating component is reduced to 3%-6% of the normal working mode.

According to the technical solution of the present invention, the capsule endoscope comprises a battery component, an image acquisition component, and a circuit processing component. The battery component is positioned between the image acquisition component and the circuit processing component.

Preferably, the battery component of the present invention is designed to be in close contact with the image acquisition component and the circuit processing component. With this design, the heat generated by the image acquisition component and the circuit processing component can be transferred to the battery component to raising the working temperature of the battery, thereby improving the performance of the battery.

Optionally, in the present invention, the outer peripheral surface of the battery component is wrapped with a coating film.

Preferably, the coating film is wrapped multiple layers around the outer peripheral surface of the battery component.

Preferably, the wrapped coating film needs to reach a certain thickness.

Preferably, the wrapped coating film is in contact with the inner wall of the capsule enclosure.

The coating film of the present invention may be polyethylene, polyvinyl chloride, or polyvinylidene chloride film. The film thickness may be 5 μm-50 μm.

In the present invention, wrapping the outer peripheral surface of the battery component with a coating film can reduce heat loss from the battery, which retains the battery heat and improves the battery performance.

Optionally, the outer peripheral surface of the battery component can be wrapped with an infrared reflective film.

Preferably, the outer peripheral surface of the battery component is wrapped with a polyethylene, polyvinyl chloride, or polyvinylidene chloride coating film and an infrared reflective film. The polyethylene, polyvinyl chloride, or polyvinylidene chloride coating film is positioned between the battery component and the infrared reflective film. The infrared reflective film is in contact with the inner wall of the capsule enclosure.

Preferably, the infrared reflective film is a polyester film.

In the present invention, wrapping infrared reflective film further reduces heat loss from the battery.

The present invention further discloses a control method for the capsule endoscope. The capsule endoscope measures the temperature of the heat-generating component in real-time and controls the heat-generating component to switch between the normal working mode and the temperature control mode based on the measured temperature signals.

Preferably, the control method for the capsule endoscope specifically includes the following steps:

(1) The capsule endoscope is controlled to work in the normal working mode in the stomach, and the circuit processing component measures the temperatures of the image acquisition component and the circuit processing component in real-time.

(2) When the temperatures of the image acquisition component and/or the circuit processing component are above the first threshold, the image acquisition component and/or the circuit processing component enter the first temperature control mode to reduce power consumption, and when the temperatures of the image acquisition component and/or the circuit processing component are equal to or below the second threshold, the image acquisition component and/or the circuit processing component are switched to the normal working mode. If the temperatures of the image acquisition component and/or the circuit processing component remain above the second threshold after continuously operating in the first temperature control mode for the first specific time, the image acquisition component and/or the circuit processing component enter the third temperature control mode to reduce power consumption, and when the temperatures of the image acquisition component and/or the circuit processing component are equal to or below the second threshold, the image acquisition component and/or the circuit processing component are switched to the normal working mode.

(3) When the capsule endoscope enters the intestinal tract, the capsule endoscope is controlled to work in the normal working mode in the intestinal tract, and the circuit processing component measures the temperatures of the image acquisition component and the circuit processing component in real-time.

(4) When the temperatures of the image acquisition component and/or the circuit processing component are above the first threshold, the image acquisition component and/or the circuit processing component enter the second temperature control mode to reduce power consumption, and when the temperatures of the image acquisition component and/or the circuit processing component are equal to or below the second threshold, the image acquisition component and/or the circuit processing component are switched to the normal working mode. If the temperatures of the image acquisition component and/or the circuit processing component remain above the second threshold after continuously operating in the second temperature control mode for the second specific time, the image acquisition component and/or the circuit processing component enter the fourth temperature control mode to reduce power consumption, and when the temperatures of the image acquisition component and/or the circuit processing component are equal to or below the second threshold, the image acquisition component and/or the circuit processing component are switched to the normal working mode.

Preferably, the control method for the capsule endoscope may also specifically includes the following steps:

(1) The capsule endoscope is controlled to work in the normal working mode in the stomach, and the circuit processing component measures the temperatures of the image acquisition component and the circuit processing component in real-time.

(2) When the temperatures of the image acquisition component and/or the circuit processing component are above the first threshold, the image acquisition component and/or the circuit processing component enter the first temperature control mode to reduce power consumption, and when the temperatures of the image acquisition component and/or the circuit processing component are equal to or below the second threshold, the image acquisition component and/or the circuit processing component are switched to the normal working mode. If the temperature of the image acquisition component or the circuit processing component remains above the second threshold after continuously operating in the first temperature control mode for the first specific time, the working position and orientation information of the capsule endoscope are recorded, and then the capsule endoscope enters the sleep state. The capsule endoscope in the sleep state is fully immersed in gastric juice for cooling or is controlled to fully immerse in the gastric juice via an external magnetic control device for cooling. When the temperatures of the image acquisition component and the circuit processing component are equal to or below the second threshold, the capsule endoscope is activated, switching the image acquisition component and the circuit processing component to the normal working mode. The capsule endoscope enters the normal working state, and returns to the original working state and continues working based on the working position and orientation information recorded before entering the sleep state.

(3) When the capsule endoscope enters the intestinal tract, the capsule endoscope is controlled to work in the normal working mode in the intestinal tract, and the circuit processing component measures the temperatures of the image acquisition component and the circuit processing component in real-time.

(4) When the temperatures of the image acquisition component and/or the circuit processing component are above the first threshold, the image acquisition component and/or the circuit processing component enter the second temperature control mode to reduce power consumption, and when the temperatures of the image acquisition component and/or the circuit processing component are equal to or below the second threshold, the image acquisition component and/or the circuit processing component are switched to the normal working mode. If the temperatures of the image acquisition component and/or the circuit processing component remain above the second threshold after continuously operating in the second temperature control mode for the second specific time, the image acquisition component and/or the circuit processing component enter the fourth temperature control mode to reduce power consumption, and when the temperatures of the image acquisition component and/or the circuit processing component are equal to or below the second threshold, the image acquisition component and/or the circuit processing component are switched to the normal working mode.

In the present invention, an artificial intelligence (AI) visual recognition method is used to determine whether the capsule endoscope has entered the intestinal tract. The AI visual recognition method is used to train on a large number of gastrointestinal images through artificial intelligence and big data technology, to establish a classification model for gastrointestinal images, and this model is used to identify whether the capsule endoscope has entered the intestinal tract.

The capsule endoscope and the control method therefor of the present invention are particularly suitable for operations with high power consumption, serving as a backup control method during high power operations. This can prevent the damage of the corresponding electronic components due to the local overheating of the heat-generating components when the capsule endoscope is working in a high power consumption state, effectively extending the battery life of the capsule endoscope while improving the examination results, and ensuring the effective operation of the capsule endoscope. In addition, wrapping the outer peripheral surface of the battery component with a coating film further improves the performance of the battery.

### BRIEF DESCRIPTION OF THE DRAWINGS

The accompanying drawings, which are included to provide a further understanding of the present invention and are incorporated in and constitute a part of the specification, in conjunction with the following embodiments of the present invention serve to explain the principle of the present invention, but do not constitute a limitation of the present invention. In the drawings:

FIG. 1 is a structural view of a capsule endoscope in accordance with the present invention.

### DETAILED DESCRIPTION

To more clearly understand the objects, features, and advantages of the present invention, a detailed description is provided below in conjunction with the accompanying drawings and specific embodiments. It should be noted that, where there is no conflict, the embodiments and features described in this application can be combined with each other.

Many specific details are set forth in the following description to provide a thorough understanding of the present invention. However, the present invention can be practiced in other ways than those specifically described herein. Therefore, the scope of the present invention is not limited by the specific embodiments disclosed below.

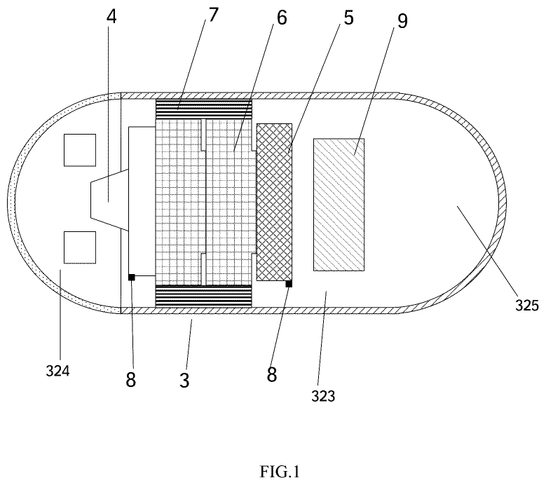

As illustrated in FIG. 1, a capsule endoscope provided comprises an enclosure (1), an image acquisition component (2), a circuit processing component (3), a battery component (4), a permanent magnet (7), and an information transmission component (not shown), among other components. The enclosure (1) comprises two ends, a first end (102) and a second end (103), and an enclosure main body (101). The image acquisition component (2) comprises or consists of an illumination (such as LED lights), a camera, and a printed circuit board (abbreviated as PCB). The circuit processing component (3) comprises or consists of a power management integrated circuit (abbreviated as PMIC), a processor, a test circuit, and a storage. The image acquisition component (2) is located at the first end (102) of the enclosure. The battery component (4) comprises or consists of two batteries. One side of the battery component (4) is in close contact with the image acquisition component (2), while the other side is in close contact with the circuit processing component (3). The outer peripheral surface of the battery component (4) is wrapped with a coating film (5), which is wrapped multiple layers until it contacts the inner wall of the enclosure (1). Temperature sensing elements (6) are disposed on both the image acquisition component (2) and the circuit processing component (3), and can be temperature transducers. The temperatures measured by the temperature sensing elements (6) are transmitted to the circuit processing component (3).

### Embodiment 1

The capsule endoscope as shown in FIG. 1 is used for stomach examination. The capsule endoscope is controlled to work in the normal working mode in the stomach. In this embodiment, the stomach examination is a high-frame-rate examination, with a frame rate of 16 frames per second. The circuit processing component measures the temperatures of the image acquisition component and the circuit processing component in real-time. When the temperatures of the image acquisition component and/or the circuit processing component are above the first threshold of 60° C., the image acquisition component and/or the circuit processing component enter the first temperature control mode to reduce power consumption. In this mode, the frame rate is adjusted to 8 frames per second to reduce the power consumption of the image acquisition component. Additionally, the storage of the circuit processing component does not perform computations, and the position detection time interval is increased, so that the power consumption is reduced to 50% of the normal working mode. When the temperatures of the image acquisition component and/or the circuit processing component are equal to or below the second threshold of 50° C., the image acquisition component and/or the circuit processing component are switched to the normal working mode. If, after remaining in the first temperature control mode for the first specified time of 30 seconds, the temperatures of the image acquisition component and/or the circuit processing component are still above 50° C., the image acquisition component and/or the circuit processing component enter the third temperature control mode to further reduce power consumption. In this mode, the frame rate is adjusted to 0.32 frames per second to further reduce the power consumption of the image acquisition component, and the circuit processing component further increases the position detection time interval, reduces the position detection accuracy, the storage does not perform computations, and the transmission power is lowered, so that the power consumption is reduced to 2% of the normal working mode. When the temperatures of the image acquisition component and/or the circuit processing component are equal to or below the second threshold of 50° C., the image acquisition component and/or the circuit processing component are switched to the normal working mode.

### Embodiment 2

The capsule endoscope as shown in FIG. 1 is used for stomach examination. The capsule endoscope is controlled to work in the normal working mode in the stomach. In this embodiment, the stomach examination is a high-frame-rate examination, with a frame rate of 25 frames per second. The circuit processing component measures the temperatures of the image acquisition component and the circuit processing component in real-time. When the temperatures of the image acquisition component and/or the circuit processing component are above the first threshold of 70° C., the image acquisition component and/or the circuit processing component enter the first temperature control mode to reduce power consumption. In this mode, the frame rate is adjusted to 10 frames per second to reduce the power consumption of the image acquisition component. Additionally, the storage of the circuit processing component does not perform computations, and the position detection time interval is increased, so that the power consumption is reduced to 40% of the normal working mode. When the temperatures of the image acquisition component and/or the circuit processing component are equal to or below the second threshold of 55° C., the image acquisition component and/or the circuit processing component are switched to the normal working mode. If, after remaining in the first temperature control mode for the first specified time of 60 seconds, the temperature of the image acquisition component or the circuit processing component is still above 55° C., the working position and orientation information of the capsule endoscope are recorded through the measurer. The capsule endoscope then enters the sleep state and is controlled via an external magnetic control device to move from outside the gastric juice to fully immerse in the gastric juice for cooling. When the temperatures of both the image acquisition component and the circuit processing component are equal to or below the second threshold of 55° C., the capsule endoscope is activated, and the image acquisition component and the circuit processing component are switched to the normal working mode. The capsule endoscope enters the normal working state, and returns to the original working state and continues working based on the recorded working position and orientation information before entering the sleep state.

### Embodiment 3

When it is determined by an AI visual recognition method that the capsule endoscope has entered the intestinal tract, the capsule endoscope is controlled to work in the normal working mode in the intestinal tract. In this embodiment, the frame rate for intestinal examination is 5 frames per second. The circuit processing component measures the temperatures of both the image acquisition component and the circuit processing component in real-time. When the temperatures of the image acquisition component and/or the circuit processing component are above the first threshold of 60° C., the image acquisition component and/or the circuit processing component enter the second temperature control mode to reduce power consumption. In this mode, the frame rate is adjusted to 4 frames per second to reduce the power consumption of the image acquisition component, and the position detection time interval is increased to reduce the power consumption of the circuit processing component, so that the power consumption is reduced to 80% of the normal working mode. When the temperatures of the image acquisition component and/or the circuit processing component are equal to or below the second threshold of 45° C., the image acquisition component and/or the circuit processing component are switched to the normal working mode. If, after remaining in the first temperature control mode for the second specified time of 10 minutes, the temperatures of the image acquisition component and/or the circuit processing component are still above 45° C., the image acquisition component and/or the circuit processing component enter the fourth temperature control mode to further reduce power consumption. In this mode, the frame rate is adjusted to 0.3 frames per second to further reduce the power consumption of the image acquisition component, and the circuit processing component further increases the position detection time interval, reduces the position detection accuracy, the storage does not perform computations, and the transmission power is lowered, so that the power consumption is reduced to 6% of the normal working mode. When the temperatures of the image acquisition component and/or the circuit processing component are equal to or below the second threshold of 45° C., the image acquisition component and/or the circuit processing component are switched to the normal working mode.

The above descriptions are only preferred embodiments of the present invention, and are not intended to limit the present invention. For those skilled in the art, the present invention may have various modifications and changes. Any modifications, equivalent replacements, improvements, etc. made within the spirit and principles of the present invention shall be included within the protection scope of the present invention.

1. A capsule endoscope, comprising a temperature sensing element, a heat-generating component, and a circuit processing component, wherein the temperature sensing element is used for measuring the temperature of the heat-generating component inside the capsule endoscope, the temperature signals measured by the temperature sensing element are transmitted to the circuit processing component, which controls the heat-generating component of the capsule endoscope to switch between a normal working mode and a temperature control mode based on the measured temperature signals.

2. The capsule endoscope of claim 1, wherein the heat-generating component comprises an image acquisition component and/or a circuit processing component.

3. The capsule endoscope of claim 1, wherein when the capsule is located in a stomach and the temperature of the heat-generating component is above a first threshold, the heat-generating component enters a first temperature control mode, and when the temperature of the heat-generating component is equal to or below a second threshold, the heat-generating component is switched to the normal working mode.

4. The capsule endoscope of claim 3, wherein if the temperature of the heat-generating component remains above the second threshold after continuously operating in the first temperature control mode for a first specific time, the heat-generating component enters a third temperature control mode, and when the temperature of the heat-generating component is equal to or below the second threshold, the heat-generating component is switched to the normal working mode.

5. The capsule endoscope of claim 4, wherein the first specific time is 30 s-60 s.

6. The capsule endoscope of claim 1, wherein when the capsule is located in the intestinal tract and the temperature of the heat-generating component is above a first threshold, the heat-generating component enters a second temperature control mode, and when the temperature of the heat-generating component is equal to or below a second threshold, the heat-generating component is switched to the normal working mode.

7. The capsule endoscope of claim 6, wherein if the temperature of the heat-generating component remains above the second threshold after continuously operating in the first temperature control mode for a second specific time, the heat-generating component enters a fourth temperature control mode, and when the temperature of the heat-generating component is equal to or below the second threshold, the heat-generating component is switched to the normal working mode.

8. The capsule endoscope of claim 7, wherein the second specific time is 1 min-10 min.

9. The capsule endoscope of claim 2, wherein the capsule endoscope comprises a battery component, an image acquisition component, and a circuit processing component, wherein the battery component is positioned between the image acquisition component and the circuit processing component.

10. A control method for the capsule endoscope, wherein the capsule endoscope measures the temperature of the heat-generating component in real-time and controls the heat-generating component to switch between a normal working mode and a temperature control mode based on the measured temperature signals.
